(19) United States
(12) Patent Application Publication
(10) Pub. No.: US 2025/0352739 A1
(43) Pub. Date: Nov. 20, 2025

(54) DOSE DIVIDER FOR A MEDICAL DEVICE

(71) Applicant: TELEFLEX MEDICAL INCORPORATED, Morrisville, NC (US)

(72) Inventor: Sophia Vorreuter, Raleigh, NC (US)

(21) Appl. No.: 19/209,273

(22) Filed: May 15, 2025

Related U.S. Application Data
(60) Provisional application No. 63/647,929, filed on May 15, 2024.

(51) Int. Cl. A61M 11/00; A61M 31/00

(52) U.S. Cl. CPC A61M 11/007; A61M 31/00; A61M 2202/0007; A61M 2202/04; A61M 2205/0216; A61M 2205/3379; A61M 2205/581; A61M 2205/582; A61M 2210/0618

(57) ABSTRACT

The invention relates to a dose divider, method, and system of the same, including a top section, a middle section, and a bottom section. The top section removably attaches to a plunger of the syringe and includes a stabilizer orient the dose divider. Further, the top section includes a first stop to prevent the top section from moving past a flange of the syringe. The middle section includes at least one projection providing feedback to a user when a unit dose of medication has been drawn up or has been administered and dosing portions separated by the projection(s). The dosing portions are configured to correspond to one unit dose of medication measured by the syringe; and. The bottom section removably attaches to a barrel of the syringe and includes a second stop to prevent the bottom section from moving past the flange of the syringe during drawing up of medication.

### CROSS-REFERENCE TO RELATED APPLICATIONS

This application is U.S. Non-Provisional application, which claims the benefit of U.S. Provisional Application No. 63/647,929, filed on May 15, 2024, entitled DOSE DIVIDER FOR A MEDICAL DEVICE, the entire content of which is incorporated by reference herein.

### FIELD OF THE INVENTION

The present invention relates to medical devices, such as a syringe, including a dose divider for administering a medication.

### BACKGROUND

Intranasal medication administration can be a non-invasive way to administer medications resulting in a quick onset of action. However, rapid, reliable, and equal dosing is important for delivering medication intranasally via atomizers. To aid in administering quick doses of medication to patients, medical professionals will often utilize dose dividers, but many dose dividers presently suffer from several problems.

First, some dose dividers require the use of both hands, with one hand placed on the dose divider and the other on the syringe to administer the medication. Often these dose dividers require the dose divider to be rotated after the administration of a first dose or removed after administration of a first dose before any additional medication can be administered. Having the dose divider require rotation or removed after a first dose before a user can administer a second dose diverts the user's attention during this process. Thus, this situation often results in unreliable, unequal, and slow delivery of medication.

Second, some dose dividers require a specific angular or spatial orientation for correct use. Because the user must align the dose divider first on the syringe before administration of medication, the user is often slow at providing a reliable dose.

Third, some dose dividers may have internal projections on the plunger of the syringe which often creates unequal and unreliable medication dosage. For example, users may push past the projections if there is not enough resistance or tactile feedback provided by the projections. Further, the projections often do not provide any indication of how many units of medication are being administered to a user. Also, these kinds of dose dividers are expensive because they require designing an entirely new plunger for a syringe rather than adapting a solution that may be attached to conventional syringes.

### SUMMARY OF THE INVENTION

The present invention relates to a dose divider for a syringe, a method of administering medicine, and a system including the dose divider, which solves the forgoing problems. For example, the present invention helps reduce manufacturing costs and facilitate equal dosing, particularly for providing two accurate and equal doses of intranasal medication one-handed without having to attach the dose divider or having to remove the dose divider between administering doses. Additionally, a system including a dose divider of the present invention comes fully assembled in a sterile package with a syringe and an atomizer so a medical practitioner may be able to use it immediately upon opening for rapid, reliable, and equal dosing.

The dose divider may include three sections: a top section, a middle section, and a bottom section. The top section may be configured to removably attach the dose divider to a plunger of the syringe and may include at least one stabilizer configured to orient the dose divider with respect to a longitudinal axis of the syringe, such as to prevent the dose divider from rotating during use. Additionally, the top section may include a first stop configured to prevent the top section from moving past a flange of the syringe when a user advances the plunger.

The middle section may at least one projection configured to provide feedback to a user when a unit dose of medication has been drawn up or has been administered; and a plurality of dosing portions separated by the at least one projection. Each of the plurality of dosing portions may be configured to correspond to one unit dose of medication measured by the syringe.

The bottom section may be configured to be removably attached to a barrel of the syringe and includes a second stop configured to prevent the bottom section from moving past the flange of the syringe when the user withdraws the plunger.

At least one stabilizer may be configured to removably attach to a top surface of the plunger. For example, the at least one stabilizer may include a pair of ridges to removably attach to a top surface of the plunger. The dose divider may also include at least one stabilizer configured to removably attach to a shaft of the plunger. For example, the at least one stabilizer may include an elastic block to removably attach to a shaft of the plunger.

The top section of the dose divider may include at least one finger pad configured to provide a grip for the user. The projection may include a first notch and a second notch configured to elastically deform. The first notch and the second notch may also be configured to prevent the projection from breaking when the projection passes over the flange.

The syringe used with the dose divider may include unit measurements for any fluid amount known to a person skilled in the art such as ounces (oz), milliliters (mL), or the like. As such, the dose divider may be used to administer one unit doses measured by the syringe of 0.25 milliliters. The first stop may include a bottom surface of the top section to prevent the top section from moving past a flange of the syringe when a user advances the plunger. The second stop may include a top surface of the bottom section to prevent the bottom section from moving past the flange of the syringe when the user withdraws the plunger. The at least one projection may provide feedback including aural, visual, and/or tactile feedback such as a sound, a click, a vibration, a pulse, tension, resistance, or otherwise identifying the at least one projection has reached or passed the flange of the syringe.

In one embodiment, the plurality of dosing portions may include only two dosing portions and the at least one projection may include only one projection. Here, the middle section may be configured to be a length corresponding to two units measured by the syringe. Further, a distance between the projection and the first stop may equal a distance between the projection and the second stop and may correspond to one unit measured by the syringe. In another embodiment, the plurality of dosing portions comprises four dosing portions and the at least one projection comprises three projections.

A method of administering medicine may use the embodiments of the dose divider as described herein. The method may include attaching the top section of the dose divider to the plunger, attaching the bottom section of the dose divider to the barrel, and inserting a tip of the syringe into the medication. A user may then withdraw the plunger to draw up the medication into the barrel of the plunger until the second stop contacts the flange of the syringe. The user may remove the tip of the syringe from the medication and place the tip of the syringe at a first treatment location. The user may then advance the plunger until the at least one projection provides feedback to administer a first dose of the medication, place the tip of the syringe at a final treatment location, and advance the plunger until the first stop contacts the flange of the syringe to administer a final dose of the medication. Feedback may include aural, visual, and/or tactile feedback such as a sound, a click, a vibration, a pulse, tension, resistance, or otherwise identifying the at least one projection has reached or passed the flange of the syringe. The user may perform the method described herein while only using one hand.

Moreover, the first treatment location may be a first nostril of a patient and the final treatment location may be a second nostril of the patient. Additionally, for each additional projection, placing the tip of the syringe at an additional treatment location and advancing the plunger until each projection contacts the flange of the syringe to administer an additional dose of the medication.

Finally, a system may use the embodiments of the dose divider as described herein including a syringe having a plunger and a barrel with a flange and a tip, and an atomizer removably attached at the tip of the syringe for administering medication.

The dose divider may include three sections: a top section, a middle section, and a bottom section. The top section may be configured to removably attach the dose divider to a plunger of the syringe and may include at least one stabilizer configured to orient the dose divider with respect to a longitudinal axis of the syringe, such as to prevent the dose divider from rotating during use. Additionally, the top section may include a first stop configured to prevent the top section from moving past a flange of the syringe when a user advances the plunger.

The middle section may at least one projection configured to provide feedback to a user when a unit dose of medication has been drawn up or has been administered; and a plurality of dosing portions separated by the at least one projection. Each of the plurality of dosing portions may be configured to correspond to one unit dose of medication measured by the syringe.

The bottom section may be configured to be removably attached to a barrel of the syringe and includes a second stop configured to prevent the bottom section from moving past the flange of the syringe when the user withdraws the plunger.

### BRIEF DESCRIPTION OF THE DRAWINGS

FIG. 1 shows an isometric view of one embodiment of a dose divider.

FIG. 2 shows a side plan view of the dose divider of FIG. 1.

FIG. 3 shows an isometric view of stabilizers of the dose divider of FIG. 1.

FIG. 4 shows a top plan view of the dose divider of FIG. 1.

FIG. 5 shows an isometric view of a system including the dose divider of FIGS. 1-4 attached to a syringe.

FIG. 6 shows a side plan view of a second embodiment of a dose divider.

FIG. 7 shows a method of administering medicine using the dose divider of FIGS. 1-4.

### DETAILED DESCRIPTION

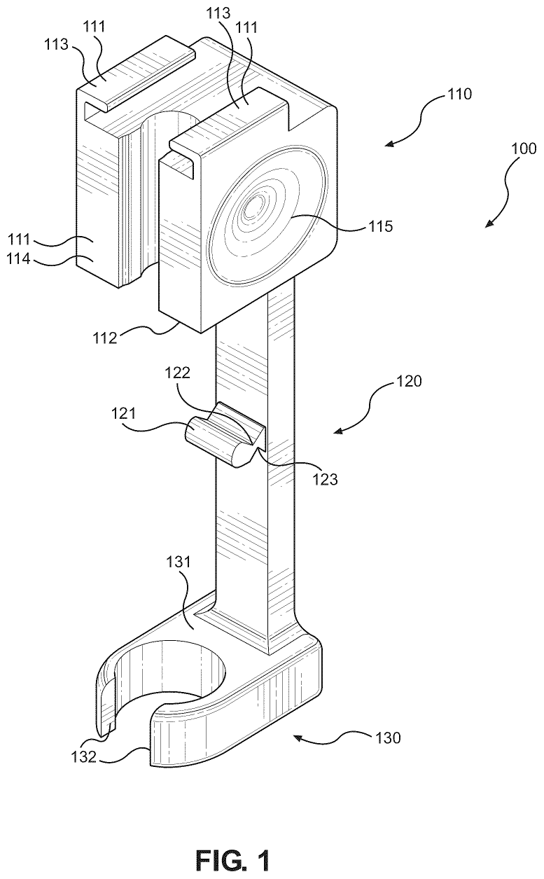

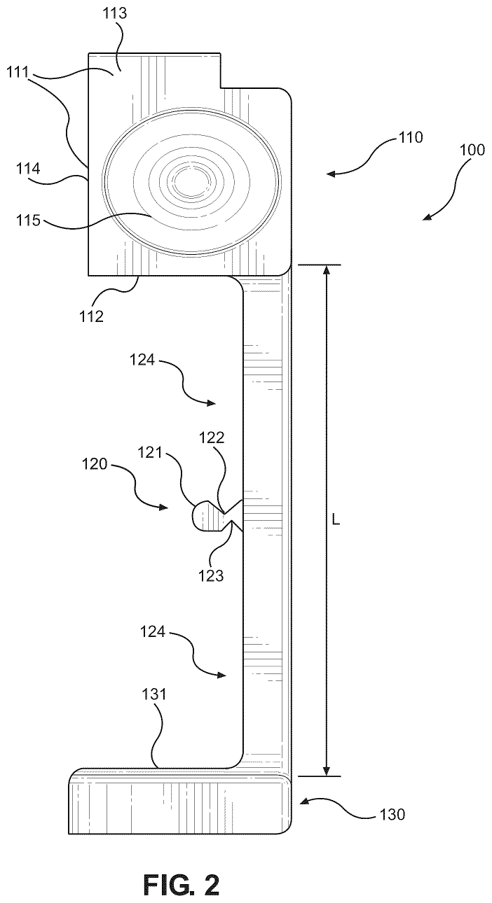

Referring to the Figures, FIGS. 1-4 disclose one embodiment a dose divider 100 used by a medical practitioner to administer two equal doses of medication. The dose divider 100 may include three sections: a top section 110, a middle section 120, and a bottom section 130. The top section 110 may be configured to removably attach the dose divider 100 to a plunger 201 of the syringe 200 and may include at least one stabilizer 111 configured to prevent rotation of the dose divider 100 around the syringe 200 and to maintain a fixed orientation in relation to the syringe 200 during use. The stabilizers 111 may include at least one feature configured to removably attach to a top surface 204 of the plunger 201 and at least one feature configured to removably attach to the shaft 205 of the plunger 201.

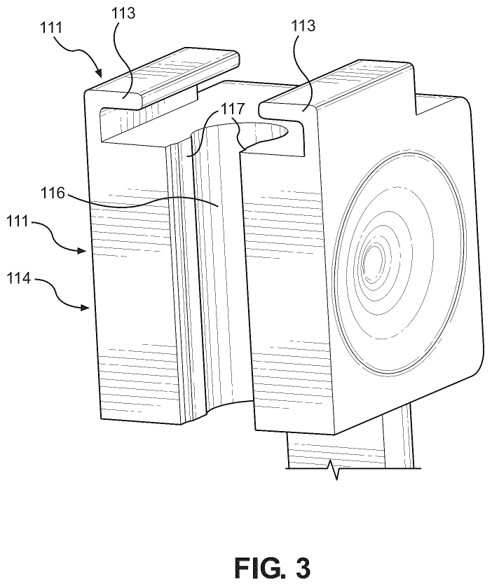

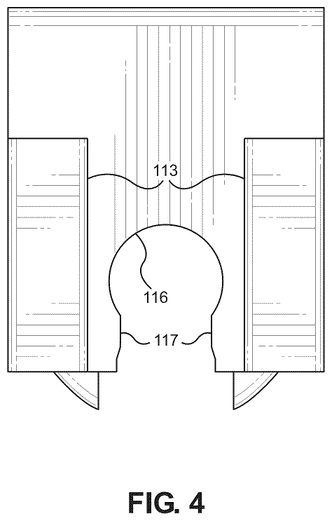

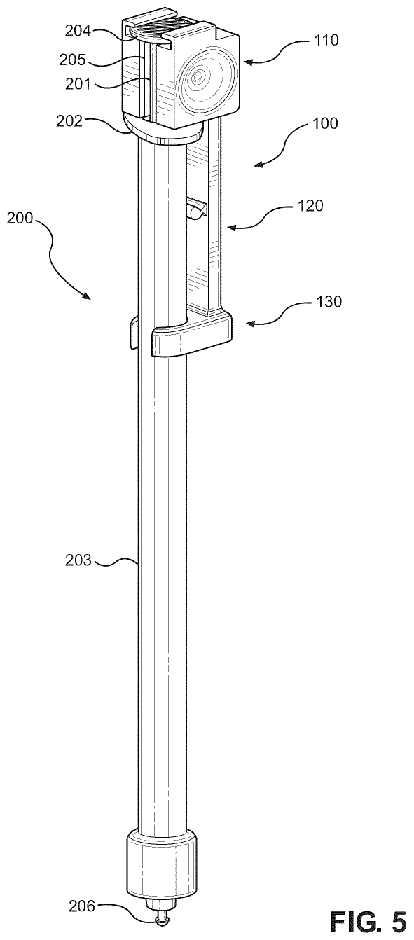

In one embodiment, the stabilizers 111 may include at least one ridge 113 which may snap, clip, grip, or otherwise removably attach to a top surface 204 of the plunger 201 as seen in FIG. 5. For example, in FIGS. 3 and 4, the stabilizers 111 may include two ridges 113 for gripping the top surface 204 of the plunger 201 and ensure the dose divider remains in a fixed angular orientation with respect to the syringe 200 during dosage administration. However, any feature such as a clip, grip, clamp, projection, protrusion, or otherwise configured to removably attach to a top surface 204 of the plunger 201 may be used to prevent rotation of the dose divider 100 and ensure a desired orientation in relation to the syringe 200 during use.

Additionally, the stabilizers 111 may include an elastic block 114 to snap onto a shaft 205 of the plunger 201. For example, in FIGS. 3 and 4, the elastic block 114 has a receiving surface 116 with a pair of resilient projections 117 which may be configured to elastically deform to receive and removably attach to the shaft 205 of the plunger 201. The receiving surface 116 is preferably cylindrical in shape but may be any shape that conforms to the shaft 205 of the plunger 200. However, any feature such as a clip, grip, clamp, projection, protrusion, or otherwise configured to removably attach to a shaft 205 of the plunger 201 may be used to prevent rotation of the dose divider 100 and ensure a desired orientation in relation to the syringe 200 during use. For example, rather than an elastic block 114, such a stabilizer 111 may have a top elastic segment and a bottom elastic segment to clip onto and conform to the shape of the shaft 205 of the plunger 200.

Further, the top section 110 of the dose divider 100 may include at least one finger pad 115 to allow a user's hand to easily grip onto the dose divider 100. In FIGS. 1-4, the top section 110 includes two finger pads 115, one on each side of the top section 110 arranged symmetrically. Preferably, these finger pads 115 form a curved or concave projection into the top section 110 of the dose divider 100. A user can then squeeze these finger pads 115 with their thumb and index finger to grip the dose divider to withdraw the plunger 201 or advance the plunger 201 with the dose divider during administration of medicine.

The top section 110 may also include a first stop 112 to prevent the top section 110 from moving past a flange 202 of the syringe 200 when a user advances the plunger 201. As seen in FIGS. 1-4, the first stop 112 may be the bottom surface of the top section 110 and preferably is flat, so that the first stop 112 becomes flush with a flange 202 of the syringe 200 after a final dose of medication is administered to a patient. However, the first stop 112 may be any surface or feature of the top section 110 of the dosage divider 100 that is configured to come into contact with the flange 202 of the syringe 200 after administration of a final dose of medication. As a result, the first stop 112 is configured to ensure the final dose is equal to the other doses administered to a patient using the dosage divider 100. Furthermore, the flange 202 may be of various shapes known to a person skilled in the art such as having an annular shape, having two wings on either side of the barrel 203 and/or the like.

In another aspect, the bottom section 130 may be configured to removably attach the dose divider 100 to a barrel 203 of the syringe 200. For example, in FIGS. 1-4, the bottom section 130 includes a pair of flexible protrusions 132 which may be configured to conform to the shape of the barrel 203 of the syringe 200. While the Figures show a cylindrical syringe 200, the barrel 203 may come in any shape known to a person skilled in the art such as quadrilateral, polygonal, and/or the like. The bottom section 130 may also include a second stop 131 to prevent the bottom section 130 from moving past the flange 202 of the syringe 200 when the user withdraws the plunger 201. As seen in FIGS. 1-4, the second stop 131 may be the top surface of the bottom section 130 and preferably is flat, so that the second stop 131 becomes flush with a flange 202 of the syringe 200 after the final dose of medication is drawn up into the barrel 203 of the syringe 200. Like the first stop 112, the second stop 131 may be any surface or feature of the bottom section 130 of the dosage divider 100 that is configured to come into contact with the flange 202 of the syringe 200 after the final dose of medication is drawn up into the syringe 200. As a result, the second stop 131 is configured to ensure the first dose administered is equal to the other doses administered to a patient using the dosage divider 100.

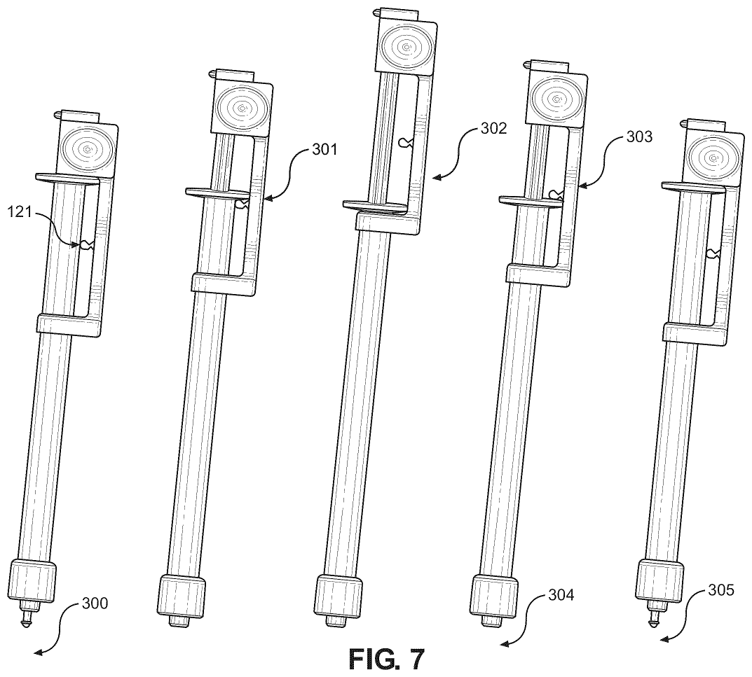

In one aspect, the middle section 120 of the dose divider 100 may have a plurality of dosing portions 124 separated by at least one projection 121. Each of these dosing portions 124 is configured to correspond to one unit dose of medication measured by the syringe. projection 121 configured to provide feedback to a user when the projection 121 contacts the flange 202 of the syringe 200. Feedback may include tactile, visual, and/or aural feedback such as a click, vibration, pulse, tension, resistance, and/or the like. For example, as seen in FIG. 7, there are two instances at which the projection 121 contacts the flange 202 of the syringe 200: a first instance 301 when a user is withdrawing the plunger and drawing up medicine into the barrel 203 of the syringe 200 and a second instance 303 immediately after a user is administers a first dose of medicine at a first treatment location 304.

At the first instance 301, the top of the projection 121 contacts the bottom surface of the flange 202 of the syringe 200 and creates tactile feedback in the form of tension, vibration, pulse, or resistance felt by the user. Feedback may also be made visually from the user inspecting the location of the dose divider 100 along the syringe 200 or by audibly hearing a clicking sound or otherwise identifying the at least one projection 121 has reached or passed the flange 202 of the syringe 200.

Preferably the projection 121 is made elastically deformable by at least one notch (122, 123) formed in the projection 121. As such, the projection 121 will elastically deform when the user provides enough force to allow the projection 121 to pass over the flange 202. Owing to this feature, the first notch 122 may also be configured to prevent the projection 121 from breaking when the projection 121 passes over the flange 202 when withdrawing or advancing the plunger 201. The tension or resistance felt by the user will then reduce when the projection 121 passes over the flange 202.

At the second instance 303, the same process occurs but in reverse. The bottom of the projection 121 contacts the top surface of the flange 202 of the syringe 200 and creates tactile feedback in the form of tension or resistance felt by the user. Again, the projection 121 will elastically deform when the user provides enough force to allow the projection 121 to pass over the flange 202. Owing to this feature, the projection 121 may have a second notch 123 also configured to prevent the projection 121 from breaking when the projection passes over the flange 202 when withdrawing or advancing the plunger 201. The tension or resistance felt by the user will then reduce when the projection 121 passes over the flange 202. This movement may further produce a sound or click which may be audible to the user.

In another aspect, the middle section 120 of the dose divider 100 may have a total length L corresponding to two units, where one unit corresponds to one dose of medication measured by the syringe 200. Further, a distance between the projection 121 and the first stop 112 and a distance between the projection 121 and the second stop 131 may correspond to one unit. In the Figures, for example, a standard 1 mL syringe 200 is utilized with the dose divider 100 for the administration of two units of medication comprising equal doses of 0.25 mL intranasal medication. Thus, the middle section 120 of the dose divider 100 is a length L that corresponds to 0.5 mL measured by the syringe 200. Additionally, the distance between the projection 121 and the first stop 112 corresponds to one unit of 0.25 mL measured by the syringe 200 and the distance between the projection 121 and the second stop 131 corresponds to one unit of 0.25 mL measured by the syringe 200. However, the syringe 200 can include unit measurements for any fluid amount known to a person skilled in the art such as ounces (oz), milliliters (mL), or the like. For example, commonly used syringes 200 may hold 1 mL or 2 mL of fluid medication and may include units measured by the syringe of 0.25 mL or 0.5 mL.

The total length L of the middle section 120 of the dose divider 100 may be based on the geometry of the syringe 200 being used. For example, standard 1 mL syringes are often cylindrical and may have a barrel diameter of around 4 to 6 mm. The volume (V) for the amount of medication drawn up by the syringe may be represented by the equation V=πr²h, where r represents the radius of the barrel of the syringe and h represents the height the plunger has been drawn up. Given this relationship, for a dose divider 100 that is used to administer two equal doses of 0.25 mL, the total length L of the middle section 120 of the dose divider will also be equal to the height (h) of the plunger has drawn up 0.5 mL or 0.5 cm³ of medication. Thus, the total length L of the middle section 120 may be between 17.7 to 39.8 mm based on the particular dimensions of the syringe 200 used.

Moreover, each dosing portion 124 of the middle section 120 may have a length similarly corresponding to one unit dose of medication measured by the syringe. As such, a dosing portion 124 corresponding to a unit dose of 0.25 mL may have a length between 8.85 to 19.9 mm. For example, the dose divider 100 of FIGS. 1-4 may have a middle section length L of 30.25 mm, a length of each dosing portion 124 may be about 15.12 mm, and a total length of the dose divider 100, including the top, middle, and bottom sections (110, 120, 130), may be 56.5 mm.

However, in other aspects, the middle section 120 of the dose divider 100 may have a length L corresponding to more than two units, such as, three, four, or more units and have a corresponding amount of dosing portions 124. Additionally, the middle section 120 of the dose divider 100 may include more than one projection 121 such as, two, three, or more projections 121. Further, a distance between the at least one projection 121 and the first stop 112, a distance between the at least one projection 121 and the second stop 131, and/or a distance between the at least one projection 121 and an adjacent projection 121 corresponds to one unit measured by the syringe 200.

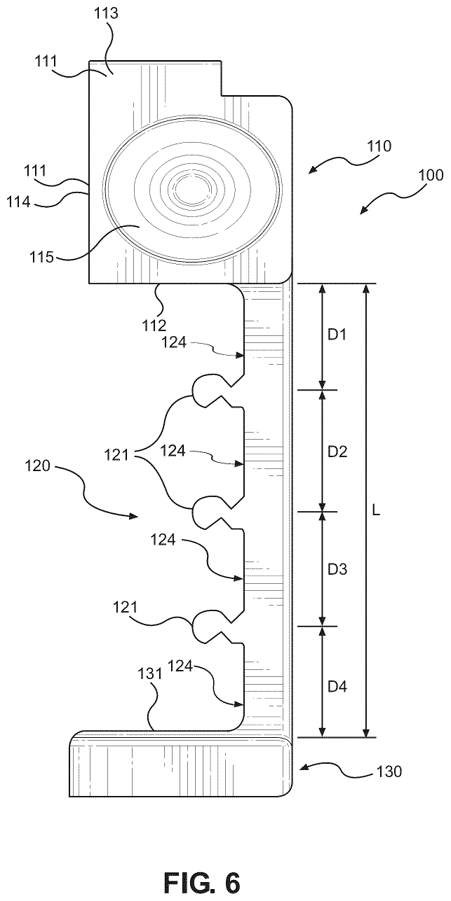

For example, FIG. 6 illustrates an embodiment of a dose divider including three projections 121 and four dosing portions 124, which allows a user to provide four equal unit doses of medication. The middle section 120 of the dose divider 100 may have a length L corresponding to four unit doses measured by the syringe 200 and each dosing portion 124 having a length corresponding to one unit dose measured by the syringe 200. In this case, the three projections 121 of the middle section 120 of the dose divider 100 are equally spaced along the length L of the middle section 120 and separate the middle section 120 into the four equal dosing portions 124. In this configuration, the distance D1 between the first stop 112 and the first projection 121 corresponds to a first dosing portion 124, the distance D2 between the first projection 121 and the second projection 121 corresponds to a second dosing portion 124, the distance D3 between the second projection 121 and the third projection 121 corresponds to a third dosing portion 124, and the distance D4 between the third projection 121 and the second stop 131 corresponds to the fourth dosing portion 124. Moreover, D1, D2, D3, and D4 are all equal and all correspond to one unit measured by the syringe 200.

Those skilled in the art will understand the number of projections 121 used and the total length L of the middle section 120 of the dose divider 100 is limited by the amount of fluid designed to be held by the syringe 200, the desired number of equal doses, and the geometry of the syringe 200. For example, if a dose divider 100 is used for a standard cylindrical 1 mL syringe 200 for 0.25 mL doses, the middle section 120 of the dose divider 100 will have at most two projections 121 with a length L of the middle section 120 corresponding to four units measured by the syringe 200 allowing for four equal doses of 0.25 mL.

The dose divider 100 may be made in one piece through manufacturing methods known to those skilled in the art such as 3D printing, injection molding, casting, and/or the like. Further the dose divider 100 may be made of plastic, acrylic, resin, or other materials known to those skilled in the art to increase efficiency and reduce cost of the dose divider 100.

FIG. 7 discloses a method of administering medicine using the dose divider 100 as described. For example, during use, a medical practitioner may attach the top section 110 of the dose divider 100 to the plunger 201 of the syringe 200, attach the bottom section 130 of the dose divider 100 to the barrel 203 of the syringe 200, and insert a tip 206 of the syringe 200 into the medication as indicated in position 300. The practitioner may then withdraw the plunger 201 along with the dose divider 100 to draw up the medication into the barrel 203 of the syringe 200.

As seen in the first instance 301, when the practitioner withdraws the plunger 201, a projection 121 of the dose divider 100 may provide tactile feedback at the flange 202 of the syringe 200 to provide indication that a dose of medication has been withdrawn into the syringe 200. The practitioner may continue to withdraw the plunger 201 until position 302, when the second stop 131 of the dose divider 100 reaches the flange 202 of the syringe 200, at which point the syringe 200 will be ready for dosage administration.

The practitioner may then remove the tip 206 of the syringe 200 from the medication, place the tip 206 of the syringe 200 at a first treatment location 304, and advance the plunger 201 until the projection 121 provides tactile feedback at the flange 202 of the syringe 200 to administer a first dose of the medication as seen at instance 303. The practitioner may then place the tip 206 of the syringe 200 at a second treatment location 305 and advance the plunger 201 until the first stop 112 contacts the flange 202 of the syringe 200 to administer a second dose of the medication.

In other aspects, the user performs the method described herein while only using one hand. Further, the first treatment location 304 may be a first nostril of a patient and the first treatment location 305 may be a second nostril of the patient. In this manner, two equal doses of medication may be administered to a patient intranasally.

The method of administering medicine may also be made to provide more than two doses in cases where the middle section 120 of the dose divider 100 has more than one projection 121 and the length L corresponds to more than two units measured by the syringe 200, such as the dose divider 100 seen in FIG. 6. Those skilled in the art will understand that each projection 121 may provide feedback when moving past the flange 202 of the syringe 200 both when drawing up the medication and by advancing the syringe 200. Moreover, each tactile feedback corresponds to a dose of medication, with the final dose being administered when the first stop 112 contacts the top surface of the flange 202 of the syringe 200 and the final dose being drawn up when the second stop 131 contacts the bottom surface of the flange 202 of the syringe 200. In such a way, the instant invention provides for a dose divider 100 and method of administering equal doses of medication quickly and reliably at a plurality of treatment locations.

FIG. 5 discloses a system which may include a syringe 200 having a plunger 201 and a barrel 203 with a flange 202 and a tip 206, a dose divider 100, and an atomizer removably attached at the tip 206 of the syringe 200 for administering medication. The atomizer used in the system may include any atomizer known to those skilled in the art such as those sold under the trademarks LMA, MAD NASAL, or VAXINATOR, including those disclosed in U.S. Pat. No. 11,241,547, the disclosure of which is hereby incorporated in its entirety. Such a system may include the dose divider 100, syringe 200, and atomizer manufactured as part of one sterile package for immediate use.

For brevity, like elements are described as in the embodiments above. In one aspect, the dose divider 100 may include a top section 110, a middle section 120, and a bottom section 130. The top section 110 may be configured to removably attach the dose divider 100 to a plunger 201 of the syringe 200 and may include at least one stabilizer 111 to prevent rotation of the dose divider 100 around the syringe 200.

Additionally, the top section 110 may include a first stop 112 to prevent the top section 110 from moving past a flange 202 of the syringe 200 when a user advances the plunger 201. Here, the middle section 120 may be configured to be a length L corresponding to two units measured by the syringe 200 and include one projection 121 to provide tactile feedback to a user when the at least one projection 121 contacts the flange 202 of the syringe 200. Additionally, the bottom section 130 may be configured to removably attach the dose divider 100 to a barrel 203 of the syringe 200 and includes a second stop 131 configured to prevent the bottom section 130 from moving past the flange 202 of the syringe 200 when the user withdraws the plunger 201. Further, a distance between the projection 121 and the first stop 112 and a distance between the projection 121 and the second stop 131 corresponds to one unit measured by the syringe 200. This system comes fully assembled in a package so a user may be able to use it immediately upon opening.

The foregoing disclosure provides illustration and description but is not intended to be exhaustive or to limit the implementations to the precise form disclosed. Modifications may be made in light of the above disclosure or may be acquired from practice of the implementations. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A dose divider for a syringe, the dose divider comprising:
a top section configured to be removably attached to a plunger of the syringe comprising:
at least one stabilizer configured to orient the dose divider with respect to a longitudinal axis of the syringe; and
a first stop configured to prevent the top section from moving past a flange of the syringe when a user advances the plunger;
a middle section comprising:
at least one projection configured to provide feedback to a user when a unit dose of medication has been drawn up or has been administered; and
a plurality of dosing portions separated by the at least one projection;
wherein each of the plurality of dosing portions are configured to correspond to one unit dose of medication measured by the syringe; and
a bottom section configured to be removably attached to a barrel of the syringe comprising:
a second stop configured to prevent the bottom section from moving past the flange of the syringe when the user withdraws the plunger.

2. The dose divider of claim 1, wherein the at least one stabilizer is configured to removably attach to a top surface of the plunger.

3. The dose divider of claim 1, wherein the at least one stabilizer is configured to removably attach to a shaft of the plunger.

4. The dose divider of claim 1, wherein the at least one stabilizer comprises a pair of ridges and an elastic block.

5. The dose divider of claim 1, wherein the top section comprises at least one finger pad configured to provide a grip for the user.

6. The dose divider of claim 1, wherein the at least one projection comprises a first notch and a second notch configured to allow the at least one projection to elastically deform.

7. The dose divider of claim 6, wherein the first and second notches are configured to prevent the at least one projection from breaking when the at least one projection passes over the flange.

8. The dose divider of claim 1, wherein one unit dose measured by the syringe is 0.25 milliliters.

9. The dose divider of claim 1, wherein the second stop comprises a top surface of the bottom section.

10. The dose divider of claim 1, wherein the first stop comprises a bottom surface of the top section.

11. The dose divider of claim 1, wherein the feedback comprises a sound, a click, a vibration, a pulse, tension, resistance, or identifying the at least one projection has reached or passed the flange of the syringe.

12. The dose divider of claim 1, wherein the plurality of dosing portions comprises two dosing portions and the at least one projection comprises one projection.

13. The dose divider of claim 12, wherein the middle section configured to be a length corresponding to two unit doses of medication measured by the syringe.

14. The dose divider of claim 12, wherein a distance between the one projection and the first stop equals a distance between the one projection and the second stop and corresponds to one unit measured by the syringe.

15. The dose divider of claim 1, wherein the plurality of dosing portions comprises four dosing portions and the at least one projection comprises three projections.

16. A method of administering medicine using the dose divider of claim 1, comprising:
attaching the top section to the plunger;
attaching the bottom section to the barrel;
inserting a tip of the syringe into a medication;
withdrawing the plunger to draw up the medication into the barrel of the plunger until the second stop contacts the flange of the syringe;
removing the tip of the syringe from the medication;
placing the tip of the syringe at a first treatment location;
advancing the plunger until the at least one projection provides feedback to administer a first dose of the medication;
placing the tip of the syringe at a final treatment location; and
advancing the plunger until the first stop contacts the flange of the syringe to administer a final dose of the medication.

17. The method of claim 16, wherein the user performs the method while only using one hand.

18. The method of claim 16, wherein the first treatment location is a first nostril of a patient and the final treatment location is a second nostril of the patient.

19. The method of claim 16, further comprising, for each additional projection, placing the tip of the syringe at an additional treatment location and advancing the plunger until each projection provides feedback to administer an additional dose of the medication.

20. A system for administering medication, comprising:
a syringe comprising:
a plunger; and
a barrel comprising
a flange and a tip;
a dose divider comprising:
a top section configured to be removably attached to a plunger of the syringe comprising
at least one stabilizer configured to orient the dose divider with respect to
a longitudinal axis of the syringe; and
a first stop configured to prevent the top section from moving past a flange of the syringe when a user advances the plunger;
a middle section comprising
at least one projection configured to provide feedback to a user when a unit dose of medication has been drawn up or has been administered; and
a plurality of dosing portions separated by the at least one projection;
wherein each of the plurality of dosing portions are configured to correspond to one unit dose of medication measured by the syringe; and
a bottom section configured to be removably attached to a barrel of the syringe comprising
a second stop configured to prevent the bottom section from moving past the flange of the syringe when the user withdraws the plunger; and
an atomizer removably attached at the tip of the syringe configured to administer atomized medication.
